(19) United States
(12) Patent Application Publication
(10) Pub. No.: US 2010/0318194 A1
(43) Pub. Date: Dec. 16, 2010

(54) Prosthesis with chargeable electric energy accumulator

(76) Inventors: Gregor Puchhammer, Wien (AT); Hubert Egger, Wien (AT)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/528,180

(22) PCT Filed: Feb. 21, 2008
(86) PCT No.: PCT/EP08/01377

§ 371 Date: Aug. 21, 2009

(30) Foreign Application Priority Data
Feb. 23, 2007 (DE) 10 2007 009 356.1

(51) Int. Cl. A61F 2/48

(52) U.S. Cl. 623/24

(57) ABSTRACT

The invention relates to a prosthesis with an electric energy accumulator (12), wherein the prosthesis comprises (a) an induction coil (28) and (b) a charging connection (24) for charging the energy accumulator (12) based on electric current induced in the induction coil (28), the connection being electrically connected to the induction coil (28) and the energy accumulator (12).

The invention relates to a prosthesis with an electrical energy store.

Modern prostheses have electrical actuators in order, for example, to move elements of the prosthesis or in order to influence the characteristics of passive elements, such as damping elements. The electrical energy required by these actuators is stored in rechargeable batteries. This rechargeable battery must be charged at regular intervals. By way of example, the rechargeable battery may be replaceable for this purpose. When the rechargeable battery in the prosthesis is dead, then the prosthesis is removed and the dead rechargeable battery is replaced by a charged rechargeable battery.

This solution has the disadvantage that the rechargeable battery must be replaced at regular intervals, which is tedious. According to one further alternative, the rechargeable battery is recharged by being connected via a charging cable to an external electrical power source. However, it has been found that this way of charging the rechargeable battery is susceptible to faults.

The invention is based on the object of proposing a prosthesis in which the rechargeable battery can be charged with less probability of faults.

The invention solves the problem by a prosthesis of this generic type which has an induction coil and a charging circuit, which is electrically connected to the induction coil and to the energy store, for charging the energy store on the basis of an electric current which is induced in the induction coil.

During the analysis of faults which occur when rechargeable batteries in prostheses are charged by means of charging cables, it has been found that contact difficulties frequently occur in the area of the plug or socket for the plug. The reason for this is that patients who, for example, are wearing an arm prosthesis can, of course, insert the plug into the socket into the prosthesis using only one hand. The prosthesis can move during this insertion process. In order to counteract this movement of the prosthesis, the plug by means of which the charging cable is connected to the prosthesis is introduced by the patient with a jerk, which places a severe mechanical load on the socket. Furthermore, the plug and socket are subject to increased wear by contact between the socket and water or dirt, which is once again compensated for by the patient by using greater force for insertion of the plug. The forces that result in this case can easily result in contact problems between the socket and its connections. A further reason that has been found for contact problems is dirt in the plug caused by an accumulation of, for example, sweat with clothing fibers, worn-off cosmetic foam, dust and the like. In order to overcome contact problems caused in this way, patients use greater force for insertion of the plug, thus loading the plug and socket.

Since the invention provides that the rechargeable battery of the prosthesis can be charged via the induction coil, this considerably reduces the mechanical load on the components involved, such as the plug, socket and associated connections. This advantageously results in a considerably longer prosthesis life.

A further advantage is that the outer casing of the prosthesis can be provided as standard with a skin-colored protective casing, which need not be interrupted by a socket. This improves the esthetic impression of the prosthesis, and increases the convenience of wear by the patient. A further advantage is that the provision of the induction coil allows the prosthesis to be encased without any interruption and therefore such that it is resistant to water spray. The prosthesis is therefore more robust and more user-friendly.

A further advantage is that, because of the presence of the induction coil, the prosthesis can be placed in a suitably shaped charging apparatus for charging. The process of connection of a plug or of some other external component can then be avoided completely, reducing the patient's sense that the prosthesis is a machine. This improves the acceptance of the prosthesis by the patient.

In particular, the prosthesis according to the invention is an exoprosthesis, which is externally accessible and can be detached from the body of the patient.

In one preferred embodiment, the induction coil is electrically connected to a load modulation circuit which is part of a common circuit with the induction coil and is designed to apply impedance modulation to the circuit. One advantage in this case is that the impedance modulation allows information to be transmitted from the prosthesis to an induction coil which interacts with the induction coil. For example, if the prosthesis has a microprocessor which detects operating cycles or fault messages from electrical components of the prosthesis, then this operating data can be transmitted via the impedance modulation.

In one preferred embodiment, the prosthesis comprises a closed water-tight outer casing. A closed water-tight outer casing means in particular that, when the prosthesis is attached to the body of the patient, no water spray can reach other components of the prosthesis. Components which are arranged in the interior of the outer casing, such as motors and microprocessors, are therefore protected against contact with water spray. This improves the operational reliability of the prosthesis. A prosthesis such as this is furthermore completely externally electrically insulated, thus protecting microelectronic components against being damaged by electrical charges. This is because externally accessible electrical contacts can carry electrostatic charges such as these which, for example, can occur when the patient pulls a pullover off, to the microelectrical components.

In order to avoid adversely affecting the mobility of the prosthesis, the induction coil is preferably flexible.

The prosthesis is preferably provided with a charging coil, which can be removed from the prosthesis, for interaction with the induction coil, thus resulting in a prosthesis system. The induction coil is in this case tuned to a natural frequency of the charging coil such that, when an alternating current is applied to the charging coil and is moved into the vicinity of the induction coil, an electric current is induced in the induction coil and electrically charges the energy store. The charging coil is connected via at least one charging cable to an electrical power source, in order to supply the charging coil with a charging current, and the electrical power source is designed to apply control signals for the prostheses to the charging current. By way of example, the control signals are applied by varying the amplitude of the charging current. Alternatively, a high-frequency electric current can be added to the charging current. It is also possible to frequency-modulate the charging current.

The electrical power source may, for example, be designed in order to transmit such control signals to the prosthesis, which codes commands in order to read operating parameters from the prosthesis.

A particularly reliable connection is maintained between the induction coil and the charging coil if the charging coil is designed for interlocking connection to the prosthesis. This can be done by the charging coil itself having an appropriate geometric shape. Alternatively, the charging coil can be surrounded by a casing, which is designed for interlocking connection to the prosthesis.

It is particularly advantageous for the prosthesis to be a hand prosthesis which comprises at least one finger, and for the charging coil to be designed to surround the finger like a ring. In this case, the charging coil can simply be pulled over one of the fingers like a jewelry ring, thus being securely fixed on the prosthesis. Alternatively, it is possible for the charging coil to be attached to a holder in the form of an arm ring which, for example, can be reversibly connected to the prosthesis by a movement radially towards a hand joint of the prosthesis.

A prosthesis system which can be operated particularly easily is obtained when the charging coil comprises a magnet which is designed for interaction with a ferromagnetic component of the induction coil, in particular with a core of the induction coil, such that the charging coil can be reversibly attached to the prosthesis on the basis of a magnetic attraction force. In this case, the charging coil just needs to be moved into the vicinity of the induction coil. The magnet then attracts the ferromagnetic core of the induction coil, and the induction coil is connected to the charging coil with a force fit. In this case, the magnet does not influence the transmission behavior of energy and data.

The magnet is particularly preferably designed such that it is released from the prosthesis by a mechanical load, before the magnet or the charging cable is damaged. For example, if the prosthesis falls down while it is connected to the charging coil, then this does not result in any damage to the prosthesis or the charging coil. In contrast, a plug and socket system according to the prior art would result in a risk of damage.

The charging coil is particularly preferably encapsulated such that it is water-tight. This makes it possible to charge the prosthesis even during work in which there is a risk of water spray.

It is possible to provide for the magnet to be an electromagnet connected to the electrical power source, with the electrical power source being designed in order to deactivate the magnet when the energy store is full. In this case, the magnet acts as a holding magnet, which is switched off when it is not required, in order to keep the induction coil and the charging coil physically close to one another. In the unactivated state, the electromagnet advantageously does not attract any ferromagnetic objects. The user can therefore determine very easily whether the charging process has or has not been completed. The state of charge of the energy store is preferably displayed via an LED indication.

In order, for example, to allow the induction coil to be placed particularly easily over a finger of the prosthesis, the induction coil is preferably flexible.

The electrical power source is preferably designed such that, when the induction coil is not located in the immediate vicinity, a magnetic alternating field which is very greatly reduced in comparison to that during the charging mode is produced at predetermined time intervals, in the order of magnitude of seconds. When this alternating field is detected by the induction coil, then the load modulation circuit changes the impedance of the circuit in a predetermined manner. The feedback of this changed impedance is detected by the electrical power source or by a controller connected to it, with switching taking place to production of a charging alternating field. The components involved are designed such that the charging alternating field is built up only when the induction coil and the charging coil are in the immediate vicinity of one another. As a safety measure, this avoids the charging alternating field causing damage to people who, for example, have heart pacemakers.

One exemplary embodiment of the invention will be explained in more detail in the following text with reference to the attached drawings, in which:

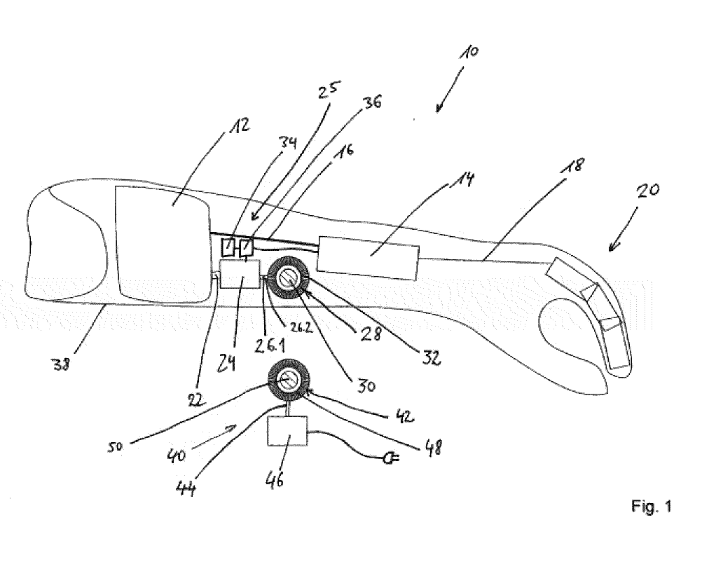

FIG. 1 shows a prosthesis system according to the invention with a prosthesis according to the invention,

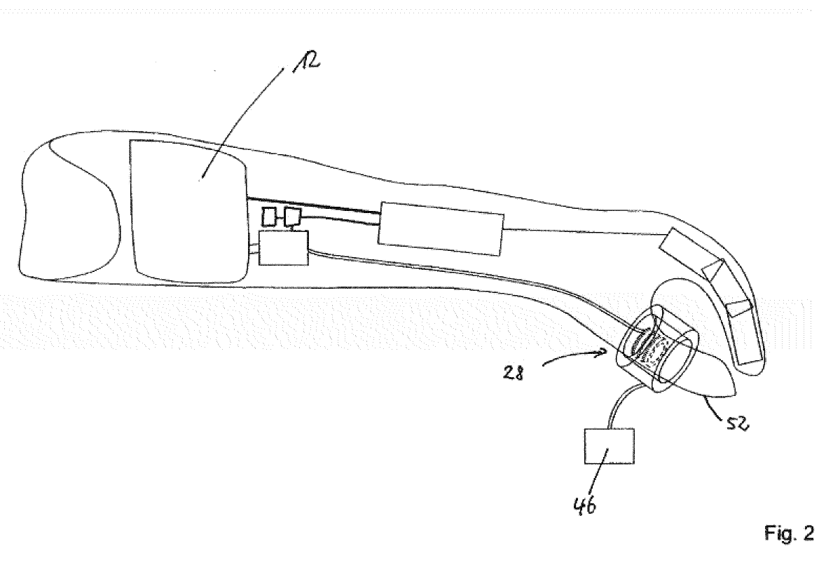

FIG. 2 shows an alternative prosthesis system, in which the charging coil is attached to a finger,

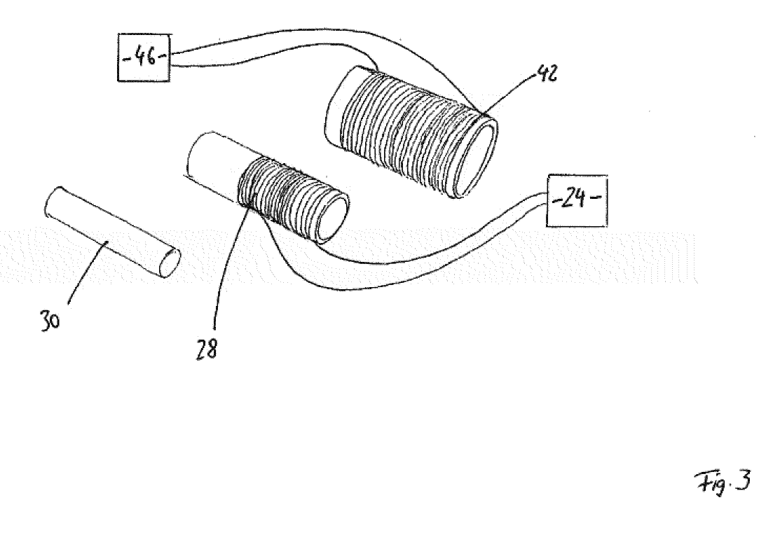

FIG. 3 shows a charging coil and an induction coil for a prosthesis according to the invention,

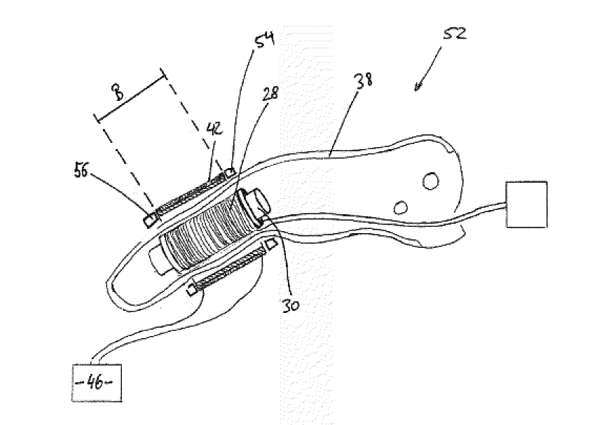

FIG. 4 shows a finger of a prosthesis according to the invention with an induction coil having a core, and

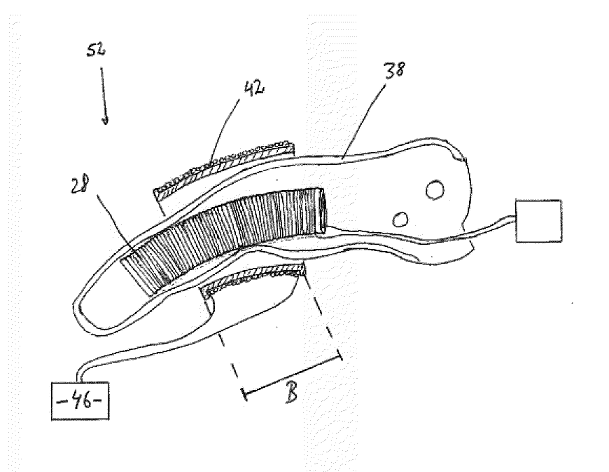

FIG. 5 shows a finger of a prosthesis according to the invention with an induction coil without a core.

FIG. 1 shows a prosthesis 10 in the form of a forearm prosthesis, which has an energy store in the form of a rechargeable battery 12, and an electric motor 14, which is connected to the rechargeable battery 12 by means of a cable 16. The electric motor 14 can operate a finger joint 20 via a pulling cable 18. Alternatively, the electric motor 14 is connected to the finger joint by means of a joint.

The rechargeable battery 12 is connected via an electrical line 22 to a charging circuit 24, which itself makes electrical contact with an induction coil 28 via an electrical wire 26. The induction coil 28 has a ferromagnetic core 30, which is arranged in the center of a winding 32.

When the induction coil 28 is placed in a magnetic alternating field, then a voltage is induced in the winding 32, which is rectified by the charging circuit 24 and is changed to a predetermined voltage. The electric current that has been rectified in this way is applied to the rechargeable battery 12, thus charging the rechargeable battery 12. The core 30 is used to increase the degree of electrical coupling of the induction coil 28.

The charging circuit 24 comprises a load modulation circuit 25 which, with the wires 26.1, 26.2 and the induction coil 28, forms a circuit. The load modulation circuit is designed to modulate the impedance of this circuit. This allows data to be transmitted to the outside world from a digital data store 34 in a microcontroller 36, which is part of the load modulation circuit 25. For example, the load modulation circuit 25 can be used to code a state of charge of the rechargeable battery 12, and to transmit this to the outside world.

The electrical components of the prosthesis 10, as described above, are surrounded by a closed water-tight outer casing 38 composed of PVC or silicone, which protects the electrical components against water spray. For prostheses in which the water-tightness is of particular importance, it is possible to provide for the outer casing 38 to be completely closed, as a result of which no electrical component becomes wet even if the prosthesis 10 is immersed in water.

The prosthesis interacts with a charger 40, which comprises a charging coil 42 which is electrically connected to an electrical power source 46 via a charging cable 44.

During operation, an alternating current flows through the charging coil 42 and produces a magnetic alternating field in the charging coil 42. When the charging coil 42 is moved sufficiently close to the induction coil 28, an alternating current is thus induced, as described above, in the induction coil 28. For this purpose, the charging coil 42 is operated with an alternating current at the resonant frequency of the induction coil 28. The charging coil 42 and the induction coil 28 are readjusted to resonance. If the coupling between the two coils changes, then the system is readjusted within a predetermined working range, such that the energy transmission and data transmission are always optional.

In order to mount the charging coil 42 in the vicinity of the induction coil 28, a magnet 50 is arranged in the center of a winding 48 of the charging coil 42. When the charging coil 42 comes into the vicinity of the induction coil 28, the magnet 50 attracts the core 30, and thus fixes the induction coil 28 and the charging coil 42 on one another. The strength of the magnet 50 is in this case chosen such that the connection between the magnet 50 and the core 30 is weaker than the mechanical connection between the charging cable 44 and the charging coil 42, such that it is detached when tension is applied to the charging cable 44, before any damage can occur.

FIG. 2 shows an alternative prosthesis system, which differs from the prosthesis system shown in FIG. 1 in that the induction coil 28 is arranged in a finger 52 in the form of a thumb. The charging coil 42 is designed such that it can be pushed over the finger 52 and can surround it like a ring. This ensures that electrical energy is transmitted to the rechargeable battery 12 in a particularly simple and operationally reliable manner.

FIG. 3 shows the charging coil 42, the induction coil 28 and the core 30 of the charging coil 28.

FIG. 4 shows a schematic view of a finger 52, which is not a thumb, and in which an induction coil 28, which has a core 30, is arranged in the area of the front two finger sections. The charging coil 72 is connected in an interlocking manner to the finger 52 by having been placed on the finger 52. The code coil 42 can be axially fixed by fitting a magnet 54, 56 to each of the two ends of the code coil 42 in such a way that the magnetic field which is built up in this case tries to align itself with the core 30. The charging coil 42 has a width B which corresponds to a distance between a first finger joint and a second finger joint of the finger 52. The width of the induction coil 28 is greater by a small amount, for example by 10 to 20%, than the width B of the charging coil 42.

FIG. 5 shows a flexible induction coil 28 without a core, whose width is more than 20% greater than the width B of the charging coil 42. The greater width makes it possible to compensate at least partially for the lack of a core 30.

### List of Reference Symbols

10 Prosthesis

12 Rechargeable battery

14 Electric motor

16 Cable

18 Pulling cable

20 Finger joint

22 Line

24 Charging circuit

25 Load modulation circuit

26.1, 26.2 Wire

28 Induction coil

30 Core

32 Winding

34 Digital data store

36 Microcontroller

38 Outer casing

40 Charger

42 Charging coil

44 Charging cable

46 Electrical power source

48 Winding

50 Magnet

52 Finger

54 Magnet

56 Magnet

B Width

1. A prosthesis with an electrical energy store (12), characterized by
(a) an induction coil (28) and
(b) a charging circuit (24), which is electrically connected to the induction coil (28) and to the energy store (12), for charging the energy store (12) on the basis of an electric current which is induced in the induction coil (28).

2. The prosthesis as claimed in claim 1, characterized by a load modulation circuit (25), which is electrically connected to the induction coil (28), is part of a common circuit with the induction coil (28) and is designed to apply impedance modulation to the circuit.

3. The prosthesis as claimed in claim 1, characterized in that the prosthesis has a closed water-tight outer casing (38).

4. The prosthesis as claimed in claim 1, characterized in that the induction coil is flexible.

5. A prosthesis system having a prosthesis (10) as claimed in claim 1, characterized by a charging coil (44), which can be removed from the prosthesis (10), for interaction with the induction coil (28).

6. The prosthesis system as claimed in claim 5, characterized in that the charging coil (42) is connected via at least one charging cable (44) to an electrical power source (46), in order to supply the charging coil (42) with a charging current, and the electrical power source (46) is designed to apply control signals for the prosthesis (10) to the charging current.

7. The prosthesis system as claimed in claim 5, characterized in that the charging coil (42) is designed for interlocking connection to the prosthesis (10).

8. The prosthesis system as claimed in claim 5, characterized in that the prosthesis (10) is a hand prosthesis which comprises at least one finger (52), and in that the charging coil (42) is designed to surround the finger (52) like a ring.

9. The prosthesis system as claimed in claim 5, characterized in that the charging coil (42) comprises a magnet (50) for interaction with a ferromagnetic component (30) of the induction coil (28), in particular with a core (30) of the induction coil (28), such that the charging coil (42) can be reversibly attached to the prosthesis (10) on the basis of a magnetic attraction force.

10. The prosthesis system as claimed in claim 9, characterized in that the magnet (50) is designed such that the magnet (50) is released from the prosthesis (10) by a mechanical load, before the magnet (50) or the at least one charging cable (44) is damaged.

11. The prosthesis system as claimed in claim 5, characterized in that the charging coil (42) is encapsulated such that it is water-tight.

12. The prosthesis system as claimed in claim 5, characterized in that
the magnet (50) is an electromagnet connected to the electrical power source (46), and
the electrical power source is designed in order to deactivate the magnet (50) when the energy store (12) is full.

13. The prosthesis system as claimed in claim 5, characterized in that the induction coil is flexible.
